(19) United States
(12) Patent Application Publication
(10) Pub. No.: US 2011/0195070 A1
(43) Pub. Date: Aug. 11, 2011

(54) DETECTION OF GRANULOSA-CELL TUMORS

(75) Inventors: David G. Huntsman, Vancouver (CA); Marco Marra, Vancouver (CA); Martin Hirst, Delta (CA); Ryan D. Morin, Vancouver (CA); Sohrab P. Shah, Vancouver (CA); Janine Senz, Vancouver (CA)

(73) Assignee: BRITISH COLUMBIA CANCER AGENCY BRANCH, Vancouver, BC (CA)

(21) Appl. No.: 13/123,313

(22) PCT Filed: Oct. 6, 2009
(86) PCT No.: PCT/CA09/01403

§ 371 Date: Apr. 8, 2011

(51) Int. Cl. A61K 39/395; C12Q 1/68; G01N 33/566; A61K 38/02; C12N 5/10; C12Q 1/02; A61P 35/00

(52) U.S. Cl. 424/139.1; 435/6.11; 436/501; 514/19.3; 435/325; 435/29

(57) ABSTRACT

A method of detecting a granulosa-cell tumor is described herein. The method involves detecting a mutation in a sample derived from a subject, indicative of substitution of tryptophan in place of cysteine at amino acid position 134 of FOXL2 protein. The mutation may be detected as a DNA mutation 402C>G in the FOXL2 gene. Methods for screening for a granulosa-cell tumor from a blood-based assay are provided, as well as methods for making a determination that an ovarian tumor is not a granulosa-cell tumor. Kits for granulosa-cell tumors are described, and a method of treating a granulosa-cell tumor are also described.

### CROSS REFERENCE TO RELATED APPLICATIONS

This application claims the benefit of priority of U.S. Provisional Patent Application No. 61/104168 filed Oct. 9, 2008, which is incorporated herein by reference in its entirety.

### FIELD OF THE INVENTION

The present invention relates generally to diagnosis and treatment of cancer. More particularly, the present invention relates to diagnostics for determining granulosa cell tumors, and to therapeutics directed against the FOXL2 genes useful for the treatment of cancer, particularly granulosa cell tumors.

### BACKGROUND OF THE INVENTION

Ovarian cancer is a significant cause of mortality in women. It is estimated that 1.4% of women born today will develop ovarian cancer. Overall this means that 1 in 72 women will develop ovarian cancer. Survival rates are poor, especially for ovarian cancer that has metastasized. Five year survival rates for ovarian cancer that has metastasised is 30.6%. Unfortunately ovarian cancer is frequently only diagnosed at advanced stages. Statistics made available from the Surveillance Epidemiology and End Results of the U.S. National Cancer Institute indicate that overall 67% are diagnosed after the cancer has metastasized and a further 7% are only diagnosed after the cancer is no longer confined to the primary site and has spread to regional lymph nodes or directly beyond the primary site.

Granulosa cell tumors are a rare gynaecological tumor type. For review, see Koukourakis et al., Integr Cancer Ther. 2008 Sep;7(3):204-15. However, granulosa cell tumors (GCTs) are the most common type of malignant ovarian sex cord-stromal tumor (SCST). The pathogenesis of these tumors is unknown. Moreover, their histopathological diagnosis can be challenging, and there is no curative treatment beyond surgery.

Granulosa tumors are typically treated with surgery but tend to recur again years after the initial diagnosis and treatment. Chemotherapy is given to patients with advanced, metastatic or recurrent disease. Although there is a long natural history to granulosa-cell tumors, the recurrence of these tumors and the limited effects of current treatments mean that there is a need for more effective diagnostic approaches, for targeted therapies, and for more effective prognostic projections.

### SUMMARY OF THE INVENTION

It is an object of the present invention to obviate or mitigate at least one disadvantage of previous method for diagnosis of granulosa tumors.

In order to identify the molecular basis of granulosa-cell tumors, a detailed molecular analysis of the genome of a number of tumors including granulosa-cell tumors was undertaken. Genetic lesions were identified that provide novel therapeutic targets, as well as diagnostic and prognostic tools for the treatment and management of cancer.

In a first aspect, the present invention provides a method of detecting a granulosa-cell tumor in a subject comprising detecting a mutation in a sample derived from a subject, the mutation being indicative of substitution of tryptophan in place of cysteine at amino acid position 134 of FOXL2 protein.

Another aspect described herein relates to a method of determining a course of treatment for ovarian cancer comprising: assessing a tumor for a mutation indicative of substitution of tryptophan in place of cysteine at amino acid position 134 of FOXL2 protein, and selecting surgery as a course of treatment when the mutation is present.

Also described herein is a method of determining that an ovarian cancer tumor is not a granulosa-cell tumor, so as to rule out this type of tumor. The method comprises assessing the tumor for a mutation indicative of substitution of tryptophan in place of cysteine at amino acid position 134 of FOXL2 protein, confirming the absence of the mutation in the tumor to determine that the tumor is not a granulosa-cell tumor.

According to an additional aspect, there is provided herein a method of screening a subject for early detection of a granulosa-cell tumor comprising testing a blood or plasma sample from the subject for the presence of a mutation indicative of substitution of tryptophan in place of cysteine at amino acid position 134 of FOXL2 protein in circulating tumor DNA or circulating tumor cells, indicative of a granulosa-cell tumor.

Further, described herein is a kit for detecting a granulosa-cell tumor in a subject, comprising: (a) reagents for sequencing DNA to locate mutation 402C>G in FOXL2, (b) a probe for selectively hybridizing to DNA comprising a 402C>G mutation in FOXL2, or (c) reagents for locating a single nucleotide polymorphism in comprising mutation 402C>G in FOXL2. The selected reagents or probe are included in the kit together with instructions for use in detecting the mutation, indicative of a granulosa-cell tumor.

There is also provided herein a kit for detecting a granulosa-cell tumor in a subject, comprising an antibody for selectively detecting a mutation in FOXL2 corresponding to C134W in SEQ ID NO:5 and instructions for use in detecting the mutation, indicative of a granulosa-cell tumor.

There is also provided herein a method of targeting a therapeutic compound to a granulosa-cell tumor comprising binding the therapeutic compound to an antibody specific for SEQ ID NO:5 at an epitope containing C134W, prior to delivery of the therapeutic compound to a subject in need thereof.

Also, a host cell transformed with a mutant FOXL2 gene comprising DNA mutation 402C>G is described herein.

Further, a method of screening agents for the ability to mediate function of FOXL2 is described. The method comprises combining the agent with a cell transformed with a mutant FOXL2 gene comprising DNA mutation 402C>G, under conditions suitable to detect FOXL2 function; and detecting a change in FOXL2 function. Agents identified with this method of screening are also encompassed.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures.

FIG. 1 illustrates data generated from whole-transcriptome paired-end RNA sequencing in Example 2.

FIG. 2 is an illustration in 3 panels, of the FOXL2 402C>G missense mutation. Panel A shows the mapped sequence reads from one of four granulosa-cell tumors. Panel B shows sequence logos representing the allele distribution of the position of the mutation and surrounding nucleotides. Panel C shows reference cDNA and protein sequences, with the mutated residues indicated by boxes.

### DETAILED DESCRIPTION

Generally, the present invention provides a method of detecting a granulosa-cell tumor in a subject. The method involves detecting a mutation in a sample derived from a subject, indicative of substitution of tryptophan in place of cysteine at amino acid position 134 of FOXL2 protein. This mutation is indicative of granulosa-cell tumors with very high specificity. There is a need to positively identify granulosa-cell tumors without using conventional means such as histological examination.

As used herein, the term “FOXL2” references the gene “forkhead box L2”. FOXL2 gene is a forkhead transcription factor that is expressed early in ovarian development. Alternative names or aliases for FOXL2 include BPES (Blepharophimosis, epicanthus inversus, and ptosis), BPES1 (Blepharophimosis, epicanthus inversus, and ptosis), PFRK (forkhead box L2), PINTO (forkhead transcription factor FOXL2), and POF3. FOXL2 may be identified in UniProtKB/Swiss-Prot as FOXL2_HUMAN, P58012 (Genecards, Weizmann Institute, Rehovot 76100, Israel). Reference to the FOXL2 protein encoded by the gene is also made throughout the specification.

As used herein, the expression “402C>G” represents the change or mutation in nucleotide bases from C to G at position 402 in the gene sequence of FOXL2, and should be understood as equivalent to the expression “402C→G”. The gene sequence of FOXL2 containing this mutation may be referenced herein as a “mutant FOXL2 gene” or missense FOXL2, as opposed to the non-mutated form having C at position 402 simply being referenced as FOXL2 gene, or as wildtype.

The expression “C134W” as used herein represents the change in amino acid residue 134 in the FOXL2 protein from cysteine or C to tryptophan or W. A FOXL2 protein containing this substitute of tryptophan for cysteine at residue 134 may be referenced herein as a “mutant FOXL2 protein”. Without this substitute residue, the protein having C at residue 134 is simply referenced as the FOXL2 protein.

The method of detecting granulosa-cell tumors described herein involves detecting a mutation in a sample derived from a subject, and determining whether the sample contains a positive indication of substitution of tryptophan in place of cysteine at amino acid position 134 of FOXL2 protein. For example, this may be determined by testing whether the mutation detected is a DNA mutation 402C>G in the FOXL2 gene. This could specifically be determined by directly detecting whether tryptophan is present in place of cysteine at position 134 of the protein encoded by the gene.

A mutation may be detected in cDNA, genomic DNA, or RNA; or may be detected as a protein mutation in FOXL2. Detecting a protein mutation in FOXL2 may involve detecting a tryptophan residue at amino acid position 134, such as is found in the sequence according to SEQ ID NO:5.

The sample in which the mutation is detected may be a sample of tumor tissue, such as may be obtained by surgery or by biopsy.

The sample in which the mutation is detected may be blood or plasma derived from a subject, in which is contained circulating tumor DNA or circulating tumor cells. One of skill in the art would know methodologies for detecting the mutation in peripheral blood, serum or plasma samples using routine testing methods. The mutation being somatic, and particular to the tumor cells, ensures that should the mutation be detected, it would be highly likely to be correlative with the presence of a granulosa-cell tumor. A subject known to have ovarian cancer may wish to have such a blood sample drawn and tested in order to make decisions about a course of treatment, as granulosa-cell tumors are known to be less responsive to drug treatment than other types of ovarian cancer. A subject who is undergoing one or more tests for cancer screening may wish to include this test with a plurality of other available tests.

The test for this mutation can be added to a complement of tests screening for various types of cancers for use in a general population not suspected to have cancer but nevertheless wishing to undergo such an assessment, in a population of cancer survivors, or in a population identified as having ovarian cancer. Further the blood or plasma testing may be conducted in a subject in recovery or remission from or ovarian cancer. Most specifically, those individuals having previously undergone surgery for removal of granulosa-cell tumors may wish to conduct such a screening test from blood or plasma periodically, as an early detection method in an effort to watch for recurrence of the disease. For those subjects previously having had a granulosa-cell tumor surgically removed, this test is particularly advantageous, as it could be a measure capable of providing highly valuable information that can impact recurrence and survival.

In the methods described herein for detecting a mutation indicative of substitution of tryptophan in place of cysteine at amino acid position 134 of FOXL2 protein, an antibody to the protein mutation in FOXL2 can be used to detect the mutation. Such an antibody may be one specifically recognizing the point of mutation. Further, sequencing methodologies may be used to detect the mutation. Alternatively, hybridization methodologies can be used to detect the mutation. All such methodologies are known to those skilled in the art.

A method of determining a course of treatment for ovarian cancer may involve assessing a tumor for a mutation indicative of substitution of tryptophan in place of cysteine at amino acid position 134 of FOXL2 protein. Assessment may involve taking a tumor biopsy, or detecting in a blood sample any circulating tumor DNA or circulating tumor cells. The selection of surgery as a course of treatment may be deemed prudent when the mutation is present. Treatment regimes specific to or known to be effective against granulosa-cell tumors could also be implemented, once a positive identification of a granulosa-cell tumor has been made.

In those instances where the method is conducted and indicates that an ovarian cancer tumor is not a granulosa-cell tumor, permits this type of cancer to be ruled out when the mutation is not present. In such cases, a course of therapy effective in combating the more common form of ovarian cancer (for example chemotherapy) could be selected.

Early detection of a granulosa-cell tumor is possible according to the invention, as a blood or plasma sample from the subject possessing circulating tumor DNA or circulating tumor cells can be tested for the presence of a mutation indicative of substitution of tryptophan in place of cysteine at amino acid position 134 of FOXL2 protein. Early detection in all forms of cancer is highly desirable and is likely to result in a more favorable outcome than when a cancer is detected at a more advanced stage.

A kit or commercial package for detecting a granulosa-cell tumor in a subject may comprise (a) reagents for sequencing DNA to locate mutation 402C>G in FOXL2, (b) a probe for selectively hybridizing to DNA comprising a 402C>G mutation in FOXL2, or (c) reagents for locating a single nucleotide polymorphism in comprising mutation 402C>G in FOXL2; and instructions for use in detecting the mutation, indicative of a granulosa-cell tumor. Further, as an embodiment of such a kit, an antibody for selectively detecting a mutation in FOXL2 corresponding to C134W in SEQ ID NO:5 could be provided.

Treating or preventing a granulosa-cell tumor in a subject may be possible by modulating mutated the FOXL2 according to SEQ ID NO:5. Modulating may involve modifying transcriptional regulation activity by mutated FOXL2 to either up-regulate or down-regulate in such a way that the likelihood of tumor growth or development is reduced. Such modulation may comprises delivery of a small molecule or peptide for modifying FOXL2 activity, or delivery of an antibody for modifying FOXL2 activity to the subject.

A method of targeting a therapeutic compound to a granulosa-cell tumor may involve binding the therapeutic compound to an antibody specific for SEQ ID NO:5 at an epitope containing C134W, prior to delivery of the therapeutic compound to a subject in need thereof.

A host cell transformed with a mutant FOXL2 gene comprising DNA mutation 402C>G may be formed according to the invention and used for further work to determine possible candidate agents for impacting disease progression in granulosa-cell tumors. A method of screening agents for the ability to mediate function of FOXL2 may involve combining the agent with a cell transformed with a mutant FOXL2 gene, which contains the DNA mutation 402C>G, under conditions suitable to detect FOXL2 function; and if a change in FOXL2 function is detected, the candidate agent may be developed further as a therapeutic composition, due to the agent's ability to mediate function of FOXL2 in the method of screening according to claim 32.

In order to identify the molecular basis of granulosa-cell tumors, a detailed molecular analysis of the genome of a number of tumors including granulosa-cell tumors was undertaken using next generation sequencing technology. Illumina™ sequencing technology (Illumina Inc., San Diego, Calif.) enables massively parallel sequencing of millions of DNA fragments (IIlumina Inc., San Diego, Calif.). DNA sequences are “read” using a sensitive fluorescence detection methodology. Read lengths are very short ˜36 by and bioinformatics methods are used to assemble the resultant data. Using this method, the genomes of a number of tumor types were extensively sequenced, including a selection of granulosa and ovarian tumors. Genetic lesions were identified that provide novel therapeutic targets, as well as diagnostic and prognostic tools for the treatment and management of cancer.

The FOXL2 gene is a forkhead transcription factor that is expressed early in ovarian development (Moumné et al., Mol Cell Endocrinol. 2008 Jan. 30;282(1-2):2-11). Because FOXL2 is mutated in granulosa-cell tumors, it is a prime candidate as key effector gene for aberrant transcriptional changes in this disease. This advantageously provides a new therapeutic target (gene/protein) for the treatment of cancer, and particularly for granulosa-cell tumors.

FOXL2 may serve as a diagnostic or predictive biomarker for risk stratification or classification of granulosa patients as well as a prognostic indicator for predicting patient response to various therapeutic regimens, including strategies to inhibit the FOXL2.

As a diagnostic biomarker, FOXL2 may serve to readily distinguish granulosa-cell tumors from other types of ovarian cancer tumors. This can be beneficial in determining an appropriate therapeutic or surgical approach to tumor removal and recurrence.

FOXL2 is an appropriate therapeutic target for treatment of ovarian cancer, and for treatment of granulosa-cell tumors, in particular. Mutated FOXL2 is an exemplary therapeutic target. In one embodiment, FOXL2 mutated at amino acid 134 with a Cysteine >Tryptophan transition serves as an exemplary therapeutic target.

Granulosa-cell tumors may be treated using appropriate pharmaceutical compositions comprising selective or non-selective small molecules directed to FOXL2, and/or mutated FOXL2. Small molecules capable of interaction with and modulation of FOXL2 may act through interruption of FOXL2-DNA binding or alternatively through mediating FOXL2 interactions with transcriptional machinery.

Granulosa-cell tumors may be treated using appropriate pharmaceutical compositions comprising nucleic acids selective for certain regions of the FOXL2 gene, and/or the mutated FOXL2 gene, and able to selectively modify the production of FOXL2 protein. Such nucleic acid-based mediating strategies to regulate FOXL2 in this setting may include, but are not limited to, RNA interference (or small-interfering RNAs, siRNA) and anti-sense technologies.

Granulosa-cell tumors may be treated using a gene therapy approach to selectively impact dominant negative forms of FOXL2 in granulosa-cell tumor cells to mediate transcriptional activation of endogenous FOXL2.

Combining standard chemotherapeutic regimens which may currently be employed in clinical practice for treatment of granulosa-cell tumors with selective FOXL2 effectors may enhance treatment efficacy, improve outcomes and reduce treatment-associated morbidity.

FOXL2 may be employed as a therapeutic target for treatment of granulosa tumors, or granulosa-type tumors arising in males as well as females, which are not ovarian cancer related.

A method of screening compounds for the ability to mediate the function of FOXL2 involves testing an agent such as an antibody, peptide, or an organic molecule (or “small molecule”) for a change in FOXL2 function in either the FOXL2 protein, or the mutated FOXL2 protein. Peptidomimetic compounds (such as peptoids) may also be screened for ability to mediate FOXL2 function. Peptidomimetic compounds include those containing non-peptidic structural elements, which are capable of mimicking or antagonizing biological action of a natural parent peptide, but which may not have classical peptide characteristics such as peptidic bonds. Candidate agents to test for the ability to mediate function of FOXL2 could be initially determined on the basis of structural features, or through rational design of molecules designed to suit a suspected or known functional region of the protein. High throughput screening of molecules from a library could also be used to determine the ability to mediate FOXL2 function. Such a method for screening may comprise combining a candidate agent for testing with a cell, such as a host cell expressing FOXL2 and/or mutant FOXL2, under conditions suitable to detect FOXL2 function, and subsequently assessing the ability of the candidate agent to mediate FOXL2 activity, indicative of either enhanced or inhibitive activity. By assessing alternative or additional parameters, such as transcriptional activation, or binding to DNA, this method for screening compounds could also identify agent able to mediate formation of the FOXL2 protein.

Granulosa-cell tumors containing mutated FOXL2 proteins may be treated with a therapeutic agent specific for mutated FOXL2 in order to reduce, reverse, or inhibit tumor growth.

Further aspects of the invention will become apparent from consideration of the ensuing description of preferred embodiments of the invention. A person skilled in the art will realize that other embodiments of the invention are possible and that the details of the invention can be modified in a number of respects, all without departing from the inventive concept. Thus, the drawings, descriptions and examples are to be regarded as illustrative in nature and not restrictive.

### EXAMPLES

### Example 1

FOXL2 Gene in Granulosa-Cell Tumor Samples

Granulosa tumor samples were compared with control ovarian carcinoma tumor samples to observe the presence of FOXL2 gene.

Methods. Using the Illumina™ sequencing platform the FOXL2 gene was sequenced in 7 granulosa tumor samples and 10 control ovarian carcinoma tumor samples. In addition the germline FOXL2 gene was sequenced in patients with granulosa-cell tumors.

Results. The identical missense point mutation was identified in 7 of the 7 granulosa-cell tumors assessed. The mutation is TGC→TGG (or “TGC>TGG”) resulting in a C→W amino acid change at position 134.

Table 1 illustrates sequence results from an initial 4 unique samples, indicating for each granulosa-cell tumor (GCT) sample, a TGC→TGG mutation was found, resulting in substitution of W for C in FOXL2. By contrast this mutation was not detected in any of the control ovarian carcinoma tumor samples. The mutation was not detected in the germline of the patients with granulosa-cell tumors.

TABLE 1
Sequence Alterations in FOXL2 Gene for Four Initial Samples^{a}

| | | descrip- | |
|---|---|---|---|
| name | notes | tion | description |
| VOA49a- | ‘0.0684931506849315 TGC | GCT | Forkhead box |
| WTSS | TGG C W CODING | | protein L2 |
| | CONSERVATIVE RADICAL’ | | |
| VOA292a- | ‘0.379310344827586 TGC | GCT | Forkhead box |
| WTSS | TGG C W CODING | | protein L2 |
| | CONSERVATIVE RADICAL’ | | |
| VOA159a- | ‘0.350649350649351 TGC | GCT | Forkhead box |
| WTSS | TGG C W CODING | | protein L2 |
| | CONSERVATIVE RADICAL’ | | |
| VOA119a- | ‘0.388888888888889 TGC | GCT | Forkhead box |
| WTSS | TGG C W CODING | | protein L2 |
| | CONSERVATIVE RADICAL’ | | |

^{a}Analysis for gene name FOXL2 on chromosome 3, Feat_type SNP, pred_method = “Ryan”, and Score 215.

In silico modeling of the effects of the FOXL2 mutation at position 134 indicates that this amino acid change will result in an activating effect on the FOXL2 transcription factor.

These data indicate the FOXL2 gene is useful for identification of granulosa tumors. FOXL2 is mutated in granulosa tumors and is thus a prime candidate as key effector gene for aberrant transcriptional changes.

### Example 2

Mutation of FOXL2 in Granulosa-Cell Tumors of the Ovary

Summary. In this example, four adult-type GCTs were analysed using whole-transcriptome paired-end RNA sequencing. Putative GCT-specific mutations were identified that were present in at least three of these samples but were absent from the transcriptomes of 11 epithelial ovarian tumors, published human genomes, and databases of single-nucleotide polymorphisms. These variants were confirmed by direct sequencing of complementary DNA and genomic DNA. Additional tumors and matched normal genomic DNA were then analysed for comparison, using a combination of direct sequencing, analyses of restriction fragment-length polymorphisms, and TaqMan assays. All four index GCTs had a missense point mutation, 402C>G (C134W), in FOXL2, a gene encoding a transcription factor known to be critical for granulosa-cell development. The FOXL2 mutation was present in 86 of 89 additional adult-type GCTs (97%), in 3 of 14 thecomas (21%), and in 1 of 10 juvenile-type GCTs (10%). The mutation was absent in 49 SCSTs of other types and in 329 unrelated ovarian or breast tumors. It was concluded that whole-transcriptome sequencing of four GCTs identified a single, recurrent somatic mutation (402C>G) in FOXL2 that was present in almost all morphologically identified adult-type GCTs. Thus, mutant FOXL2 is a potential driver in the pathogenesis of adult-type GCTs, and is a useful indicator of granulosa-cell tumors from a diagnostic viewpoint. Details of this study are outlined below.

Methods

Patients and Samples. Four ovarian adult-type GCTs obtained from patients were selected (1 primary and 3 recurrent tumors), along with 10 ovarian carcinomas and 1 cell line derived from a serous borderline tumor provided by the OvCaRe (Ovarian Cancer Research) frozen tumor bank (Vancouver, Canada). Patients provided written informed consent for research using these tumor samples before undergoing surgery. For primary validation, 74 formalin-fixed, paraffin-embedded blocks of frozen samples were obtained from additional putative GCTs, along with 48 matched samples of normal tissue. Two additional blocks (GCT78 and GCT59) were obtained that were recurrences of samples GCT29 and GCT76, respectively, which were also genotyped. In addition, frozen tissue samples from 149 epithelial ovarian tumors and 180 breast carcinomas were analysed. Another series of formalin-fixed, paraffin-embedded ovarian sex cord-stromal tumors (SCSTs) were obtained for comparative analysis. This second series consisted of 95 tumor samples: 27 adult type GCTs, 8 juvenile-type GCTs, 23 fibromas, 14 Sertoli-Leydig cell tumors, 13 thecomas, and 10 steroid-cell tumors.

Pathological Review. All tumor samples included in this Example were independently reviewed by a gynecologic pathologist before mutational analysis. In cases in which the review diagnosis differed from the source diagnosis, the samples were further reviewed by another gynecologic pathologist, who acted as arbiter. Both pathologists were unaware of the results of genomics studies. Immunohistochemical staining for calretinin, epithelial membrane antigen, and inhibin were performed. Histologic images of all GCTs were obtained with the use of a ScanScope XT digital scanning system (Aperio Technologies, Vista, Calif.). The typical histopathological feature of GCTs, uniform nuclei with variable nuclear grooves, were visible in samples obtained from the tumors subjected to whole-transcriptome paired-end RNA sequencing (using hematoxylin and eosin).

Paired-End RNA Sequencing and Analysis. RNA sequencing and subsequent data analysis was conducted. Double-stranded complementary DNA (cDNA) was synthesized from polyadenylated RNA and sheared. The fraction from 190 to 210 by was isolated and amplified with 10 cycles of a polymerase-chain-reaction (PCR) assay, according to the paired-end protocol for the Genome Analyzer II ™ (GAII) ((Illumina Inc., San Diego Calif.). The resulting libraries were then sequenced on the GAII. Short DNA sequences (reads) obtained from the GAII were mapped to the reference human genome (National Center for Biotechnology Information build 36.1, hg18) and a database of known exon junctions with the use of MAQ™ software (Redmond, Wash.) in paired-end mode. Putative point mutations and small insertions and deletions were identified. These mutations were cross-referenced against human genome databases to eliminate previously described germ-line variants. Genome instability of the index samples was determined with the use of Affymetrix 6.0™ (Affymetrix, Santa Clara, Calif.) genotyping arrays interpreted for copy number.

Mutation Validation. Variants present in at least three of the four GCT RNA-sequencing libraries were selected from RNA-sequencing libraries from 10 non-GCT ovarian tumors and from a serous borderline tumor-derived cell line for the presence of the GCT-derived variants. Transcriptome variants that were absent in the 11 non-GCT ovarian tumors were classified as GCT-specific variants and subjected to further analysis. Identification of the GCT-specific variants was confirmed using Sanger sequencing of PCR amplicons of cDNA and genomic DNA (gDNA) obtained from the index samples (both tumor and matched normal tissue when available). A combination of direct sequencing, analyses of restriction-fragment-length polymorphisms (RFLPs), and a TaqMan real-time PCR-based allelic discrimination assay (Applied Biosystems) was used to genotype the FOXL2 402C>G mutation in additional cancers. In the primary validation studies, samples were scored as positive or negative for this mutation only if there were clear and concordant results from at least two assays. The use of assays with different primers was intended to minimize PCR artifacts caused by amplifying poor-quality DNA templates from formalin-fixed, paraffin-embedded tissue blocks.

The TaqMan and RFLP assays produced clear and concordant results for all 149 non-GCT tumors, as well as for the 4 index GCT samples, and these results matched the results from sequencing of the 74 samples with interpretable results from all assays. In cases in which the PCR-RFLP analysis did not produce a PCR product of sufficient quality and quantity for further analysis (presumably because of poor-quality template DNA), it was replaced by the TaqMan assay, which was also used in primary screening for the additional series of SCSTs studied to determine the frequency of mutations in related tumors.

Results

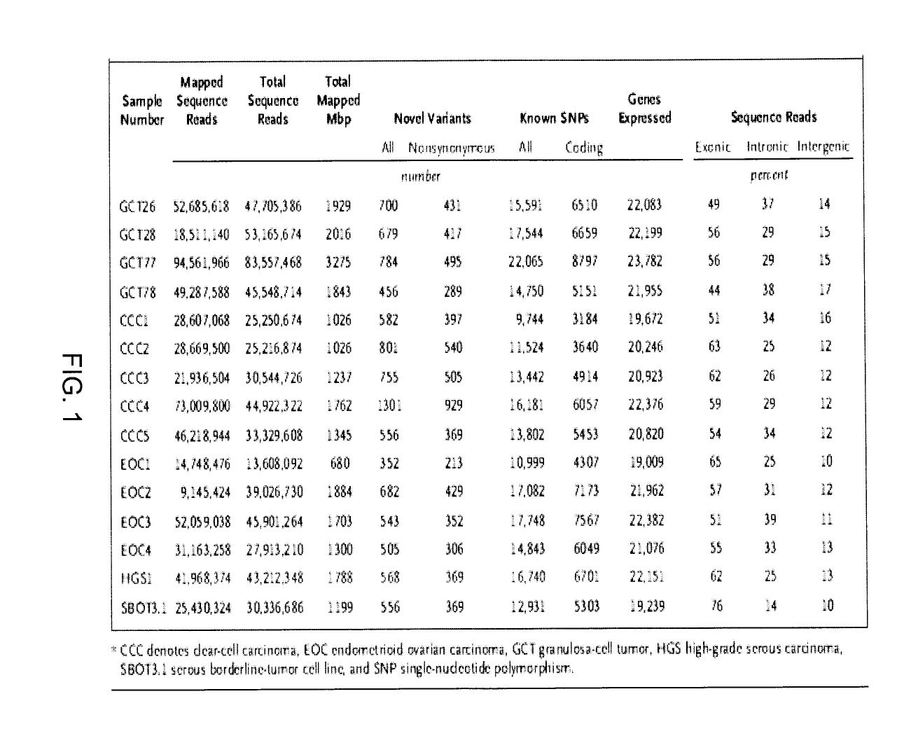

Paired-End Whole-Transcriptome RNA Sequencing. The genomic stability of the four index GCTs was confirmed by inferring DNA copy-number alterations from high-density genotyping arrays. The samples were then subjected to RNA sequencing. The total number of mapped DNA sequence reads and the total number of reads (which included the fraction of exonic, intronic, and intergenic sequences), the number of base pairs covered, and details regarding the variants detected in the 4 GCT samples and in the other 11 ovarian neoplasm-derived comparator libraries are shown in FIG. 1. Sequence reads from these samples (short strings of decoded nucleotides obtained from a single fragment of DNA or RNA to determine allelic frequency at each position) showed expected subtype-specific differential gene expression.

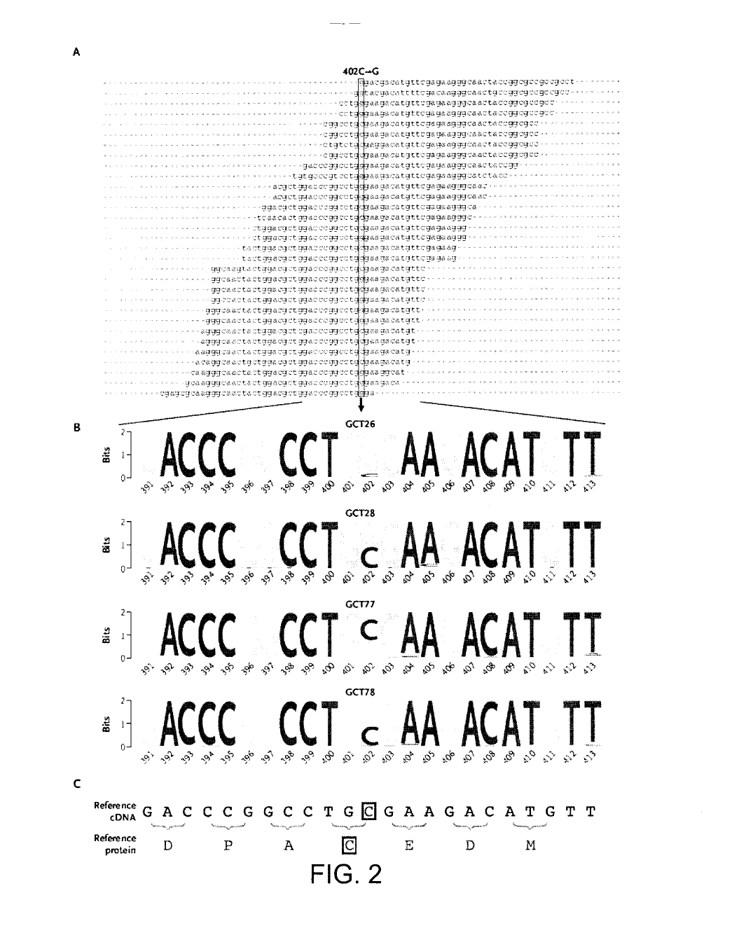

Validation of Implicated Variants. The RNA-sequencing analysis generates short reads that map to a reference sequence. FIG. 2 is an illustration in 3 panels, of the FOXL2 402C>G missense mutation.

Panel A of FIG. 2 shows the mapped sequence reads from one of four granulosa-cell tumors (GCT28) on chromosome 3 for genomic positions 140147803 to 140147903. The sequence illustrated, nnnnnnnn nnncgagcgc aagggcaact actggacgct ggacccggcc tgggacgaca tgttcgagaa gggcaactac cggcgccgcc gcctnnnnnn nnn (SEQ ID NO: 1) in FIG. 2 illustrates position 402 at genomic position 140147853 on chromosome 3; National Center for Biotechnology Information [NCBI] human genome build 36.1). In this panel, the complementary DNA (cDNA) position for FOXL2 402 is outlined as a central column, illustrating the nonreference “G” alleles.

Panel A depicts typical RNA-sequencing data for a portion of the FOXL2 gene and shows how nucleotides for expressed genes can be represented by multiple reads, as opposed to an average read generated by standard sequencing. It was predicted that the identification of mutations using SNVmix, a probabilistic model that is used to infer such a result on the basis of a binomial mixture model. Two putative point mutations that were implicated by SNVmix and a base-pair insertion met our criteria for candidate tumorigenic mutations, since they were present in three or more GCTs and absent in the comparator sequence libraries (i.e., sequence libraries generated from non-GCT tumors, in addition to sequences in existing data banks).

Panel B shows sequence logos representing the allele distribution of the position of the mutation and surrounding nucleotides. A measure of 2 bits represents the homozygous position. A bit is a measure of the purity of the distribution of alleles at each nucleotide. The variant 402C>G is clearly visible in each logo. All four GCTs assessed contained a C→G change in the FOXL2 gene at position 402 (genomic position 140147853 on chromosome 3; National Center for Biotechnology Information [NCBI] human genome build 36.1).

Panel C shows reference cDNA and protein sequences, with the mutated residues indicated by red boxes. For the mutant protein, the cysteine (C) as encoded by TGC of the reference protein is replaced by tryptophan (W), as encoded by TGG. In each of the four GCTs assessed, the C→G change in the FOXL2 gene at position 402 (genomic position 140147853 on chromosome 3; National Center for Biotechnology Information [NCBI] human genome build 36.1), would result in the substitution of a tryptophan residue in this illustrated segment DPAWEDM (SEQ ID NO:2) for a highly conserved cysteine residue at amino acid position 134, in the normal segment illustrated as DPACEDM (SEQ ID NO:3). This substitution is herein referred to as (C134W).

The FOXL2 protein is found as UniProtKB/Swiss-Prot accession number P58012 for FOXL2_HUMAN, and the function ascribed is a probable transcriptional regulator. Table N shows the sequence of FOXL2 both without the mutation C134W, and with the mutation C134W.

TABLE 2
FOXL2 Sequences With and Without C134W Mutation*
Without Mutation (SEQ ID NO: 4)

| | |
|---|---|
| 1 | mmasypeped aagallapet grtvkepegp ppspgkgggg gggtapekpd paqkppysyv |
| 61 | aliamaires aekrltlsgi yqyiiakfpf yeknkkgwqn sirhnlslne cfikvpregg |
| 121 | gerkgnywtl dpacedmfek gnyrrrrrmk rpfrpppahf qpgkglfgag gaaggcgvag |
| 181 | agadgygyla ppkylqsgfl nnswplpqpp spmpyascqm aaaaaaaaaa aaaagpgspg |
| 241 | aaavvkglag paasygpytr vqsmalppgv vnsynglggp paappppphp hphphahhlh |
| 301 | aaaapppapp hhgaaapppg qlspaspata appapaptsa pglqfacarq pelammhcsy |
| 361 | wdhdsktgal hsrldl |

With Mutation (SEQ ID NO: 5)

| | |
|---|---|
| 1 | mmasypeped aagallapet grtvkepegp ppspgkgggg gggtapekpd paqkppysyv |
| 61 | aliamaires aekrltlsgi yqyiiakfpf yeknkkgwqn sirhnlslne cfikvpregg |
| 121 | gerkgnywtl dpawedmfek gnyrrrrrmk rpfrpppahf qpgkglfgag gaaggcgvag |
| 181 | agadgygyla ppkylqsgfl nnswplpqpp spmpyascqm aaaaaaaaaa aaaagpgspg |
| 241 | aaavvkglag paasygpytr vqsmalppgv vnsynglggp paappppphp hphphahhlh |
| 301 | aaaapppapp hhgaaapppg qlspaspata appapaptsa pglqfacarq pelammhcsy |
| 361 | wdhdsktgal hsrldl |

*Swiss-Prot P58012

The Grantham distance is a measure of amino acid dissimilarity developed by Grantham in 1974 on the basis of composition, polarity, and molecular volume of amino acids. The Grantham distance of this amino acid change C→G in the FOXL2 gene at position 402 was 215, the highest value observed in the 6410 nonsynonymous predicted variants derived from the analysis of all 15 ovarian cancers outlined in FIG. 1. Of the 6410 mutations, only 15 had a Grantham distance of 215. All predicted variants from the four index GCTs are available from the European Genotyping Archive (accession number, EGA000- 0 0000040). Using two additional independent methods the FOXL2 402C>G variant was validated in the cDNA and gDNA of the four index samples and determined that it was somatic in the two samples from patients for whom normal tissue was available. The two other variants that met the criteria were one at genomic position 357452 on chromosome 4 in ZNF141 and a base-pair insertion at position 24488600 on chromosome 16 in RBBP6. Neither variant could be confirmed by PCR validation and thus they were considered to be systematic artifacts generated by misalignment of the reads. Thus the focus remained on the FOXL2 402C>G variant.

Prevalence of FOXL2 402C>G. A combination of RFLP, TaqMan, and direct-sequencing analyses was used to determine whether the FOXL2 variant was present in 69 samples of pathologically confirmed GCTs (67 adult-type and 2 juvenile-type GCTs) and three other SCSTs. Unequivocal results were obtained in 64 of 69 GCTs and in all 3 SCSTs. FOXL2 402C>G was present in 59 of 64 GCTs. Two additional paired recurrences also carried the 402C>G variant. The mutation was absent in DNA extracted from paired normal tissue from 48 mutation-positive patients. Five GCTs were mutation-negative, including the two juvenile-type GCTs (GCT24 and GCT45). Two mutation-negative tumors (GCT11 and GCT22) did not express inhibin or calretinin, which were expressed in 100% and 86% of the mutation-positive GCTs, respectively. The fifth mutation-negative tumor (GCT33) had a prominent fibrothecomatous component, also seen in two mutation-positive samples (GCT44 and GCT71). The FOXL2 mutation was absent in 149 other epithelial ovarian tumors and in 180 breast cancers. Sample GCT18, a thecoma, was the only non-GCT tumor that was positive for the FOXL2 variant in the first validation series. Further analysis of this sample revealed a minor granulosa-cell component.

The validation process conducted for non-GCT ovarian tumors and other cancers revealed that the FOXL2 402C>G mutation was confined to sex cord-stromal tumors (SCSTs), of the ovary, of which granulosa-cell tumors are the most common type. Of these tumors, 86 of 89 adult-type GCTs (97%) and 1 of 10 juvenile-type GCTs (10%) carried the mutation, as compared with none of 149 epithelial ovarian tumors and 180 breast cancers. These analyses show a high degree of specificity for the mutation in SCSTs.

Validation of the FOXL2 402C>G mutation was conducted with the use of a TaqMan allelic discrimination assay, showing a clear division between samples that were hemizygous or homozygous for the mutation (presumably through chromosome-based loss of heterozygosity), samples that were heterozygous for the mutation, and samples that were not granulosa-cell tumors (GCTs). Direct-sequencing results were obtained for matched normal tissue, GCTs that were heterozygous for the FOXL2 mutation, and for sample GCT9 that appeared to be hemizygous or homozygous for the mutation. Immunohistochemical analysis showed that FOXL2 is expressed in the nuclei of normal granulosa cells, as well as in GCTs that are either heterozygous or presumed to be hemizygous or homozygous for the 402C>G mutation. The staining pattern was normal in all cases, and the mutation did not appear to have affected nuclear localization of the FOXL2 protein.

Three tumors (GCT9, GCT35, and GCT38) were either homozygous for the 402C>G (C134W) mutation, as determined by sequencing and the TaqMan assay profile, or had a loss of heterozygosity of the normal allele. A single GCT (GCT44) had both the 402C>G (C134W) mutation and a second FOXL2 somatic variant, 404A>G (E135G). By sequencing cloned PCR products, it was observed that these variants were in cis. This sample tested positive for the 402C>G variant on RFLP analysis, but repeated TaqMan assays did not produce results. The 404 A>G variant was not detected in any of the 4 index samples, in 37 other GCTs, or in 21 non-GCTs for which sequencing data was obtained.

Fluorescence was carried out by in situ hybridization to assess potential amplification of FOXL2 on 32 GCTs and 5 epithelial ovarian cancers and obtained negative results. lmmunohistochemical analysis showed that FOXL2 was expressed in the nuclei of normal granulosa cells as well as in GCTs that were heterozygous or appeared to be hemizygous or homozygous for the 402C>G mutation.

Test for Replication. A second series of 95 clinical samples was obtained to determine the specificity of this variant within SCSTs of the ovary. It was found that FOXL2 mutation appeared in all 27 adult-type GCTs tested. Among the other ovarian SCSTs, 1 of 8 juvenile-type GCTs and 2 of 13 thecomas carried the FOXL2 variant; all other tumors (14 Sertoli-Leydig cell tumors, 23 fibromas, and 10 steroidcell tumors) were negative for the mutation.

Discussion

A recurrent somatic mutation (402C>G) was found in FOXL2 in tumor samples from four patients with GCTs, using RNA sequencing to study the transcriptomes of the samples. FOXL2 was not expressed in the other RNA-sequencing libraries derived from ovarian cancers, and the mutation was absent in the gDNA of these cancers. Two additional series of GCTs were analysed for this mutation. The combined results from both series showed that the mutation was present in 86 of 89 morphologically identified adult-type GCTs (97%), in 1 of 10 juvenile-type GCTs (10%), and in 3 of 14 thecomas (21%); the mutation was absent in 49 other ovarian SCSTs, in 149 epithelial ovarian tumors, and in 180 breast cancers.

Although juvenile-type GCTs share some features with adult-type GCTs and have a similar biomarker-expression profile, they differ with respect to clinical presentation (in prepubertal children vs. young adults) and histopathological features. These features, along with those of a naturally occurring mouse model of juvenile-type GCT, suggest that juvenile-type GCT is a distinct disease from the adult type. The data provided in this Experiment support this conclusion. Three mutation-negative GCTs would have been excluded from the study had immunostaining profiles been taken into consideration, due to their immunochistochemical profiles being distinct from most GCTs, suggesting that they were not true GCTs, but rather morphologic mimics. GCTs were primarily selected for this study on the basis of light microscopy, reflecting current diagnostic practice.

FOXL2 is a member of the forkhead-winged helix family of transcription factors containing a highly conserved DNA-binding forkhead domain. It is one of the earliest markers of ovarian differentiation, and its expression persists into adulthood. FOXL2 is required for the normal development of granulosa cells and shows strong expression in granulosa cells and moderate expression in stromal cells; no expression has been detected in oocytes. Few targets of FOXL2 have been described; it has been shown to have a role, as part of an AP1-SMAD3-SMAD4 complex, in activating the transcription of GNRHR (encoding the gonadotropin-releasing hormone receptor) in pituitary cells and repressing the transcription of TAR (encoding steroidogenic acute regulatory protein) in the adult ovary. Granulosa-cell proliferation and differentiation are governed, at least in part, by transforming growth factor βreceptor signaling through SMAD2 and SMAD3.

To date, all mutations that have been described in FOXL2 are germ-line loss-of-function mutations and are associated with the blepharophimosis-ptosis-epicanthus inversus syndrome, with primary ovarian failure (in particular, granulosa-cell failure. Like JAK2 mutations in the case of polycythemia vera, mutations in FOXL2 are present in a large majority of adult-type granulosa-cell tumors and involve a single base substitution. Cys 134 is located on the surface of the forkhead DNA binding domain; modeling suggests that the substitution of tryptophan for cysteine does not disrupt the folding of this domain or its interactions with DNA, and thus pathogenesis may be imparted through other mechanisms, such as the altering of one or more interactions between FOXL2 and other proteins. It is unlikely that mislocalization of the mutant protein is a cause of its pathogenicity, as FOXL2 is present in the nuclei of GCTs that are either heterozygous or appear to be hemizygous or homozygous for the 402C>G mutation.

Next-generation sequencing has been used to characterize mutations and gene expression in cell lines and to identify mutations in patients with acute myeloid leukemia. Some have suggested that the genomic complexity of cancers is so extreme that whole-genome-sequencing studies must be performed at great depth and must include several hundred cancers of any type to yield data that can be interpreted clinically or biologically. However, the data of this Example suggest that for some tumors (most likely the cytogenetically simple and clinicopathologically homogeneous ones, such as GCTs), the pattern of somatic mutations is recurrent and constrained and thus can be analyzed by studying a small number of samples. Since transcriptomes are less complex and 50 to 100 times smaller than genomes, sequencing transcriptomes, as opposed to genomes, has been proposed as a more efficient method of finding mutations expressed through RNA. The data in the Example supports this view. This approach cannot detect certain types of mutations, such as noncoding changes and mutations that are subject to nonsense-mediated decay.

The diagnosis of GCTs can be difficult, and treatment for this cancer is nonspecific and often unsuccessful. By testing for the presence of the FOXL2 402C>G mutation as a diagnostic indicator of GCTs, this can lead to positive identification, and more targeted therapies. Insight into the cause of this disease can be derived from knowledge of the function of this mutation.

### Example 3

The Specificity of the FOXL2 402C>G Somatic Mutation: A Survey of Solid Tumors.

Summary. In this Example, the somatic mutation in the FOXL2 gene which was present in almost all (97%; 86/89) morphologically defined adult-type granulosa-cell tumors in Example 2 was investigated further for specificity. This FOXL2 402C>G mutation changes a highly conserved cysteine residue to a tryptophan (C134W). It was also found in a minority of other ovarian malignant stromal tumors but not in benign ovarian stromal tumors or unrelated ovarian tumors or breast cancers. In this Example, other cancers and cell lines were assessed for the presence of this mutation. DNA from 752 tumors of epithelial and mesenchymal origin and 28 ovarian cancer cell lines and 52 other cancer cell lines of varied origin were screened. The FOXL2 402C>G mutation was found in an unreported A-GCT case and the A-GCT-derived cell line KGN. All other tumors and cell lines analyzed were mutation negative. In addition to proving that the KGN cell line is a useful model to study adult-type GCTs, these data show that the c. 402C>G mutation in FOXL2 is not commonly found in a wide variety of other cancers and therefore it is likely pathognomonic for GCTs and closely related tumors.

Methods

To determine the specificity of this somatic mutation, high resolution melting or polymerase chain reaction (PCR)-based allelic discrimination was used to screen a diverse collection of tumors and ovarian tumor cell lines. Additional cytogenetic analysis was performed to demonstrate the stable karyotype of the A-GCT cell line, KGN, developed by Nishi et al., 2001.

Table 3 shows the seven hundred and fifty-two tumor DNA samples, of epithelial and mesenchymal origin that were screened with a high resolution melting assay run on the LightScanner™ instrument (Idaho Technology Inc., Salt Lake City, Utah). For each tumor block, malignant cells composed >50% of the cellularity and matched normal adjacent tissue was available for all cases. The assay was designed to detect sequence variants in the region from IIe102 to Phe138 in FOXL2 (NP_{—}075555.1), which would encompass the mutation of interest at position 134.

TABLE 3
Summary of Tumor Types Screened by High Resolution
Melt Curve Analysis (HRM).

| | | | FOXL2 |
|---|---|---|---|
| | | | c.402C > G |
| | | True | mutation |
| | Total cases | positives | (Positive/ |
| | n = 752^{a} | HRM | Sequenced) |
| Ovarian cancer negative control | 14 | 0/11 | *0/3 |
| Ovarian GCT positive controls^{b} | 13 | 13/13 | 13/13 |
| Bladder Cancer | 40 | 0/40 | |
| Breast Cancer | 74 | 0/71 | *0/3 |
| Carcinoid Cancer | 8 | 0/8 | |
| Cervical Cancer | 16 | 0/16 | |
| Colorectal Cancer | 77 | 0/75 | *0/2 |
| Endometrial Cancer | 12 | 0/12 | |
| Esophageal Cancer | 21 | 0/21 | |
| Gastric Cancer | 90 | 0/89 | *0/1 |
| Head & Neck Cancer | 28 | 0/26 | *0/2 |
| Hepatic | 14 | 0/14 | |
| Lung Cancer (All types) | 125 | 0/123 | *0/2 |
| Melanoma | 31 | 0/31 | |
| Ovarian Cancer | 32 | 0/32 | |
| Pancreatic Cancer | 4 | 0/4 | |
| Prostate Cancer | 37 | 0/37 | |
| Renal Cancer | 52 | 0/51 | *0/1 |
| Sarcoma (All types) | 30 | 0/30 | |
| Testicular Cancer | 19 | 0/18 | *0/1 |
| Thyroid Cancer | 42 | 0/42 | |

^{a}Excluding Controls,
^{b}(including an unreported A-GCT case and the A-GCT cell line, KGN),
*variants seen on HRM screen that could not be ruled out by the second HRM screen

Since FOXL2 is a single exon gene, PCR primers were placed in the coding region, with the forward primer 5′-AGAAGGGCTGGCAAAATAGC (SEQ ID NO:6) , and the reverse primer 5′-GCCGGTAGTTGCCCTTCT (SEQ ID NO:7), resulting in a 150 base pair amplicon.

The primary screen used whole genome amplified (Qiagen Repli-G™ kit, Quiagen, Germantown, Md.) DNA derived from frozen tissue blocks of untreated primary tumors. All samples which had an aberrant melting curve or which failed to amplify in the initial screen were followed up with a repeat HRM assay using unamplified DNA prepared from tumor and adjacent normal tissue. Tumor samples which were repeat positive for an aberrant melting curve were sequenced in duplicate, and the resulting sequence trace files were analyzed for mutations using the phrap/phred/consed software package (www.phrap.org). DNA from 27 ovarian tumor samples previously genotyped for the mutation using a previously validated TaqMan real-time PCR-based allelic discrimination assay (Applied Biosystems, Foster City, Calif.) specific for the FOXL2 c. 402C>G mutation were used to validate the performance of the HRM assay. This included an unreported A-GCT case and the cell line KGN.

To establish the specificity of the FOXL2 c. 402C>G mutation in ovarian cancer cell lines, the same TaqMan real-time PCR-based allelic discrimination assay was used to genotype 28 ovarian cancer cell lines and 52 cancer cell lines of different tissue origin for the FOXL2 c. 402C>G mutation.

To assess the cytogenic profile of KGN, a 24-color fluorescence in situ hybridization (FISH) was used (24XCyte, MetaSystems, Cat. D-0125-120-MC) and analyzed the results using the Axioplan™ 2, Zeiss (MetaSystems, Isis), camera VAC-30054.

Results

All 11 previously reported FOXL2 c. 402C>G mutation-positive A-GCT specimens as well as an unreported A-GCT case and the A-GCT cell line, KGN, validated the HRM assay by demonstrating a variant melt curve distinct from the common (wildtype) pattern. None of the 14 FOXL2 c. 402C>G mutation negative samples exhibited this variant melt profile. Although three of the 10 high grade serous ovarian cancers showed an alternative variant profile; sequencing confirmed them to be false positives.

The primary screen of 752 whole genome amplified tumor DNA samples yielded 24 samples (˜4%) with a variant profile, distinct from that seen in association with the FOXL2 c. 402C>G mutation-positive A-GCT specimens, as well as 41 with an indeterminate profile and 29 samples that failed. The secondary screen was performed on this set of 94 samples using unamplified genomic DNA derived from tumors and matching normal specimens. Eighty-two of the samples were found to be false positives where there was no variant profile seen between the tumor and normal DNA. Twelve of the samples remained indeterminate and were subsequently sequenced and confirmed to be false positives.

The granulosa-cell line KGN, which was derived through long-term passage of a recurrent A-GCT, was the only cell line found to harbor the mutation. The mutation was not present in an SVOG granulosa-cell line, immortalized by SV40, or 26 other ovarian cancer derived cell lines. Unlike most ovarian cancer derived cell lines KGN showed relative genomic stability. In addition to deletion of 7q, it is monosomic for chromosome 22 which is the most frequent cytogenetic abnormality seen in A-GCTs; another feature demonstrating its similarities to A-GCTs.

Discussion

The specificity evidenced in this Example indicates that FOXL2 402C>G is an appropriate marker for diagnosis of granulosa-cell tumors.

Loss-of-function germline mutations in FOXL2 are associated with blepharophimosis-ptosis-epicanthus-inversus syndrome (BPES;OMIM#110100) ;a developmental disorder characterized by eyelid malformations and premature ovarian failure due to a dysfunction of granulosa-cells. Given that most A-GCTs are heterozygous for the mutation, suggests that the mutation either acts as a gain-of-function or a dominant-negative. Immunohistochemical data indicating that FOXL2 expression of these tumors is maintained in the nuclei, implies that this mutation does not function as a dominant-negative. Therefore as opposed to loss-of-function mutations causing granulosa-cell failure, in cancer of the same cells, the characteristic FOXL2 c. 402C>G mutation may lead to a gain or change of function that is ultimately oncogenic.

Analysis of 28 various ovarian cancer-derived cell lines demonstrated that the mutation was only present in the granulosa-cell tumor cell line, KGN, suggesting that it is molecularly akin to A-GCTs. The presence of the missense mutation in the well-characterized A-GCT cell line, KGN, is in keeping with the high frequency of the somatic mutation in A-GCTs and supports the use of this cell model to study the properties of this ovarian sex cord stromal tumor. However without understanding the consequences of this particular mutation, this discovery leads to the consideration of possible confounding effects of the missense mutation in studies utilizing the KGN cell line to examine FOXL2 function and the effects of FOXL2 missense, haploinsufficient or hypomorphic mutations associated with BPES.

The absence of the FOXL2 c. 402C>G mutation in this large series of common epithelial malignancies such as lung, colorectal, breast, gastric, bladder, thyroid, prostate, melanoma and ovarian carcinoma, in addition to a range of less frequent tumors, implies a high specificity of this recurrent mutation for ovarian sex cord stromal tumors, and particularly granulosa-cell tumors. The mutation was not found in non-GCT ovarian tumor cell lines and the SV40 transformed granulosa-cell line, SVOG, provides further support of its likely role in granulosa-cell tumor disease initiation. Considering the extremely high frequency of this mutation in morphologically selected GCTs (97%), these data provide further evidence that the mutation is specific for granulosa-cell tumors and would be useful as a diagnostic test. Further studies will be required to determine the relevance of the mutation in other sex cord stromal tumors of the ovary, however, it is possible that all mutation positive tumors could ultimately be considered to be a single entity of which the major component would be A-GCTs.

In the preceding description, for purposes of explanation, numerous details are set forth in order to provide a thorough understanding of the embodiments of the invention. However, it will be apparent to one skilled in the art that these specific details are not required in order to practice the invention.

The above-described embodiments of the invention are intended to be examples only. Alterations, modifications and variations can be effected to the particular embodiments by those of skill in the art without departing from the scope of the invention, which is defined solely by the claims appended hereto.

### REFERENCES

Koukourakis GV, et al., Integr Cancer Ther. 2008 Sep;7(3):204-15.

Moumné et al., Mol Cell Endocrinol. 2008 Jan 30;282(1-2):2-11.

Nishi et al. Endocrinology 2001; 142(1): 437-445.

All documents referred to herein are incorporated by reference.

1. A method of detecting a granulosa-cell tumor in a subject comprising detecting a mutation in a sample derived from a subject, indicative of substitution of tryptophan in place of cysteine at amino acid position 134 of FOXL2 protein.

2. The method of claim 1 wherein:
the mutation detected is a DNA mutation 402C>G in the FOXL2 gene; or
the mutation detected is a DNA mutation leading to substitution of tryptophan in place of cysteine at position 134 of the encoded protein.

3. (canceled)

4. The method of claim 1, wherein detecting a mutation comprises:
detecting a mutation in cDNA, genomic DNA, or RNA; or
detecting a protein mutation in FOXL2.

5. The method of claim 4, wherein detecting a protein mutation in FOXL2 comprises detecting a tryptophan residue at amino acid position 134 according to SEQ ID NO:5.

6. (canceled)

7. The method of claim 1 wherein:
the sample is a tumor tissue sample obtained by surgery or by biopsy; or
the sample is blood or plasma containing circulating tumor DNA or circulating tumor cells.

8. (canceled)

9. The method of claim 7 wherein:
the sample of blood or plasma is drawn from a subject for cancer screening; or
the sample of blood or plasma is drawn from a subject in recovery or remission from or ovarian cancer.

10. (canceled)

11. The method of claim 7 wherein the subject has previously had a granulosa-cell tumor surgically removed.

12. The method of claim 1 wherein:
an antibody to the protein mutation in FOXL2 is used to detect the mutation; or
sequencing or hybridization is used to detect the mutation.

13. (canceled)

14. (canceled)

15. A method of determining a course of treatment for ovarian cancer comprising:
assessing a tumor for a mutation indicative of substitution of tryptophan in place of cysteine at amino acid position 134 of FOXL2 protein, and selecting surgery as a course of treatment when the mutation is present; or determining that the tumor is not a granulosa-cell tumor when the mutation is absent.

16. The method of claim 15, wherein the mutation comprises a DNA mutation 402C>G in FOXL2.

17. (canceled)

18. (canceled)

19. (canceled)

20. (canceled)

21. A method of screening a subject for early detection of a granulosa-cell tumor comprising testing a blood or plasma sample from the subject for the presence of a mutation indicative of substitution of tryptophan in place of cysteine at amino acid position 134 of FOXL2 protein in circulating tumor DNA or circulating tumor cells, indicative of a granulosa-cell tumor.

22. The method of claim 21, wherein the mutation comprises a DNA mutation 402C>G in FOXL2.

23. (canceled)

24. A kit for detecting a granulosa-cell tumor in a subject, comprising:
(a) reagents for sequencing DNA to locate mutation 402C>G in FOXL2, (b) a probe for selectively hybridizing to DNA comprising a 402C>G mutation in FOXL2, or (c) reagents for locating a single nucleotide polymorphism in comprising mutation 402C>G in FOXL2; and
instructions for use in detecting the mutation, indicative of a granulosa-cell tumor.

25. The kit according to claim 24 for detecting a granulosa-cell tumor in a subject, comprising:
an antibody for selectively detecting a mutation in FOXL2 corresponding to C134W in SEQ ID NO:5, and
instructions for use in detecting the mutation, indicative of a granulosa-cell tumor.

26. A method of treating or preventing a granulosa-cell tumor in a subject comprising modulating mutated FOXL2 according to SEQ ID NO:5.

27. The method of claim 26, wherein modulating comprises modifying transcriptional regulation activity by mutated FOXL2.

28. The method of claim 26, wherein modulating comprises delivery of a small molecule or peptide for modifying FOXL2 activity, or delivery of an antibody for modifying FOXL2 activity to the subject.

29. The method of claim 26, wherein modulating mutated FOXL2 comprises targeting a therapeutic compound to a granulosa-cell tumor comprising binding the therapeutic compound to an antibody specific for SEQ ID NO:5 at an epitope containing C134W, prior to delivery of the therapeutic compound to a subject in need thereof.

30. A host cell transformed with a mutant FOXL2 gene comprising DNA mutation 402C>G.

31. A method of screening agents for the ability to mediate function of FOXL2 comprising: combining the agent with a cell according to claim 30, transformed with a mutant FOXL2 gene comprising DNA mutation 402C>G, under conditions suitable to detect FOXL2 function; and detecting a change in FOXL2 function.

32. (canceled)


SEQUENCE LISTING (7 sequences)

SEQ ID NO: 1 - DNA, 101 bp, Homo sapiens
  misc_feature (1)..(11): n is a, c, g, or t
nnnnnnnnnnncgagcgcaagggcaactactggacgctggacccggcctgggacgacatgttcgagaagggcaactaccggcgccgccgcctnnnnnnnnn

SEQ ID NO: 2 - protein, 7 aa, Homo sapiens
DPAWEDM

SEQ ID NO: 3 - protein, 7 aa, Homo sapiens
DPACEDM

SEQ ID NO: 4 - protein, 376 aa, Homo sapiens
  MISC_FEATURE (134)..(134): Cysteine residue of interest in mutation C134W
MMASYPEPEDAAGALLAPETGRTVKEPEGPPPSPGKGGGGGGGTAPEKPDPAQKPPYSYVALIAMAIRESAEKRLTLSGIYQYIIAKFPFYEKNKKGWQNSIRHNLSLNECFIKVPREGGGERKGNYWTLDPACEDMFEKGNYRRRRRMKRPFRPPPAHFQPGKGLFGAGGAAGGCGVAGAGADGYGYLAPPKYLQSGFLNNSWPLPQPPSPMPYASCQMAAAAAAAAAAAAAAGPGSPGAAAVVKGLAGPAASYGPYTRVQSMALPPGVVNSYNGLGGPPAAPPPPPHPHPHPHAHHLHAAAAPPPAPPHHGAAAPPPGQLSPASPATAAPPAPAPTSAPGLQFACARQPELAMMHCSYWDHDSKTGALHSRLDL

SEQ ID NO: 5 - protein, 376 aa, Homo sapiens
  MISC_FEATURE (134)..(134): Tryptophan residue of interest in C134W
MMASYPEPEDAAGALLAPETGRTVKEPEGPPPSPGKGGGGGGGTAPEKPDPAQKPPYSYVALIAMAIRESAEKRLTLSGIYQYIIAKFPFYEKNKKGWQNSIRHNLSLNECFIKVPREGGGERKGNYWTLDPAWEDMFEKGNYRRRRRMKRPFRPPPAHFQPGKGLFGAGGAAGGCGVAGAGADGYGYLAPPKYLQSGFLNNSWPLPQPPSPMPYASCQMAAAAAAAAAAAAAAGPGSPGAAAVVKGLAGPAASYGPYTRVQSMALPPGVVNSYNGLGGPPAAPPPPPHPHPHPHAHHLHAAAAPPPAPPHHGAAAPPPGQLSPASPATAAPPAPAPTSAPGLQFACARQPELAMMHCSYWDHDSKTGALHSRLDL

SEQ ID NO: 6 - DNA, 20 bp, Artificial Sequence
  Forward primer FOXL2
agaagggctggcaaaatagc

SEQ ID NO: 7 - DNA, 18 bp, Artificial Sequence
  Reverse primer FOXL2
gccggtagttgcccttct